(19) United States
(12) Patent Application Publication
(10) Pub. No.: US 2026/0210923 A1
(43) Pub. Date: Jul. 23, 2026

(54) RECESSED SMOKE DETECTOR

(71) Applicant: Microchip Technology Incorporated, Chandler, AZ (US)

(72) Inventors: Arthur B. Eck, Gilbert, AZ (US); Patrick McFarland, Gilbert, AZ (US)

(73) Assignee: Microchip Technology Incorporated, Chandler, AZ (US)

(21) Appl. No.: 19/093,983

(22) Filed: Mar. 28, 2025

Related U.S. Application Data
(60) Provisional application No. 63/748,700, filed on Jan. 23, 2025.

(51) Int. Cl. G01N 33/00; G01D 11/24

(52) U.S. Cl. CPC G01N 33/0027; G01D 11/245

(57) ABSTRACT

An apparatus may have a detector configured to fit substantially within a utility box installed in a cavity of a ceiling or a wall in a room, a wiring assembly configured to couple the detector to a power source, and an attachment assembly configured to attach the detector to the utility box, wherein the detector may be recessed within the utility box when attached to the utility box.

### PRIORITY

This application claims priority to commonly owned United States Patent Application No. 63/748,700 filed January 23, 2025, the entire contents of which are hereby incorporated by reference for all purposes.

### FIELD OF THE INVENTION

The present disclosure relates to monitoring systems and methods. Various examples of the teachings herein include systems and/or methods for recessing monitoring systems (e.g., smoke detectors, carbon monoxide detectors, among others) in pre-existing cavities, e.g., a utility box in a room’s wall or ceiling.

### BACKGROUND

Currently available monitoring systems and sensors, e.g., carbon monoxide and smoke detectors, have become a standard safety feature in residential and commercial buildings, playing a crucial role in early fire and carbon monoxide detection and prevention of loss of life and property damage. These devices are typically mounted on ceilings or walls to monitor the air for the presence of smoke particles and carbon monoxide, triggering an alarm when one or both are detected.

The installation of these monitoring systems is often regulated by building codes and safety standards, which specify requirements for their placement, interconnection, and power sources. Due to these building codes and regulations, most homes built in the last 40 years include monitoring systems, e.g., smoke detectors, that are interconnected and mounted on the same kind of utility box that is used for the home’s outlets and switches.

Traditional monitoring systems are often designed as standalone units that protrude from the surface they are mounted on. While functional, these protruding designs can be visually obtrusive and may not align well with interior design aesthetics.

Thus, monitoring systems that can seamlessly integrate into living spaces without compromising safety, functionality, or aesthetics is needed. This disclosure presents a monitoring system, e.g., smoke detector, with a form factor that fits within a utility box so that it does not appear as an unsightly protrusion. Fitting within an existing utility box reuses the utility box, simplifies installation of the monitoring system, and allows for the addition of an attractive, paintable cover.

### SUMMARY

The examples herein enable systems and methods for recessing monitoring systems (e.g., smoke detectors, carbon monoxide detectors, among others) in pre-existing cavities in an aesthetically pleasing way.

According to one example, an apparatus is provided that includes a detector which may be configured to fit substantially within a utility box installed in a cavity of a ceiling or a wall in a room; a wiring assembly which may be configured to couple the detector to a power source; and an attachment assembly which may be configured to attach the detector to the utility box, wherein the detector may be recessed within the utility box when attached to the utility box. In an example, the detector may be a smoke detector, a carbon monoxide detector, or both. In the same or different examples, the apparatus may include a cover configured to attach to the utility box, wherein a surface of the cover may be configured to be substantially flush with a ceiling surface of the ceiling or a wall surface of the wall when the cover is attached to the utility box. In the same or different examples, the cover may include one or more openings to allow airflow from the room into the utility box. In the same or different examples, the cover may include a gasket configured to form a seal between the cover and the utility box. In the same or different examples, the cover may include a plurality of holes configured to align with a plurality of pre-existing screw holes in the utility box. In the same or different examples, the cover may be detachable from the utility box. In the same or different examples, the attachment assembly may include a plurality of holes configured to align with a plurality of pre-existing screw holes in the utility box. In the same or different examples, the attachment assembly may include a clip configured to clip onto the utility box.

According to another example, a system is provided that includes a utility box installed in a cavity of a ceiling or a wall in a room, and a first monitoring system that may be configured to fit substantially within the utility box. In an example, the first monitoring system may include at least one of a smoke detector and a carbon monoxide detector. The first monitoring system according to this example may include a wiring assembly that may be configured to couple the first monitoring system to a power source, an attachment assembly that may be configured to attach the first monitoring system to the utility box, and a cover that may include one or more openings to allow airflow from the room into the utility box. In the same or different example, the first monitoring system may be recessed within the utility box when attached to the utility box. In the same or different examples, the wiring assembly may be configured to couple the first monitoring system to a second monitoring system. In the same or different examples, the attachment assembly may include a plurality of holes configured to align with a plurality of pre-existing screw holes in the utility box. In the same or different examples, the attachment assembly may include a clip configured to clip onto the utility box. In the same or different examples, the cover may be configured to (1) detachably attach to one of the first monitoring system, the ceiling, the wall, and the utility box; (2) form a seal between the cover and the utility box; and (3) overlap and be substantially flush with a ceiling surface of the ceiling or a wall surface of the wall when attached to the one of the first monitoring system, the ceiling, the wall, and the utility box. In the same or different examples, a first surface of the cover may be in contact with the ceiling surface of the wall surface. In the same or different examples, the first surface of the cover may form a substantially continuous seal with the ceiling surface or the wall surface. In the same or different examples, the cover may include a plurality of holes that may be configured to align with a plurality of pre-existing screw holes in the utility box.

Another example provides a method which may include placing a first monitoring system into a utility box installed in a cavity of a ceiling or a wall in a room, the first monitoring system fitting substantially within the utility box and being recessed within the utility box; coupling the first monitoring system to a power source; attaching the first monitoring system to the utility box; and attaching a cover over the cavity and the first monitoring system, the cover may form a seal between the cover and the utility box and between the cover and the ceiling or the wall. In the same or different examples, the attaching the first monitoring system to the utility box may include using pre-existing screw holes in the utility box to secure the first monitoring system to the utility box. In the same or different examples, the attaching the cover over the cavity and the first monitoring system may include snapping the cover onto one of the utility box and the first monitoring system.

### BRIEF DESCRIPTION OF THE DRAWINGS

The figures illustrate example systems and methods for recessing monitoring systems (e.g., smoke detectors, carbon monoxide detectors, among others) in pre-existing cavities in an aesthetically pleasing way.

FIG. 1 illustrates a block diagram of an example apparatus for recessing monitoring systems.

FIG. 2 illustrates a block diagram of an example apparatus for recessing monitoring systems.

FIG. 3 illustrates a block diagram of an example apparatus for recessing monitoring systems.

FIG. 4 illustrates a block diagram of an example apparatus for recessing monitoring systems.

FIG. 5 illustrates various aspects of an example apparatus for recessing monitoring systems.

FIG. 6 illustrates various aspects of an example apparatus for recessing monitoring systems.

FIG. 7 illustrates a block diagram of an example apparatus for recessing monitoring systems.

FIG. 8 illustrates various aspects of example apparatuses for recessing monitoring systems.

FIG. 9 illustrates a block diagram of an example apparatus for recessing monitoring systems.

FIG. 10 illustrates various aspects of an example apparatus for recessing monitoring systems.

FIG. 11 illustrates a block diagram of an example system for recessing monitoring systems.

FIG. 12 illustrates a flow chart of an example method for recessing monitoring systems.

FIG. 13 illustrates a flow chart of an example method for recessing monitoring systems.

The reference number for any illustrated element that appears in multiple different figures has the same meaning across the multiple figures, and the mention or discussion herein of any illustrated element in the context of any particular figure also applies to each other figure, if any, in which that same illustrated element is shown.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

The teachings of the present disclosure may be employed to reduce and/or eliminate the unsightliness of a monitoring system, e.g., a smoke detector, a carbon monoxide detector, among others. In some examples, the teachings include systems and/or methods to recess a monitoring system into a pre-existing, standard utility box found in an existing building, e.g., a home. In other examples, the monitoring system may be recessed into a standard utility box for installation in a new building. In the same or different examples, the monitoring system may include a cover. In some examples, the cover includes voids (e.g., holes) so that air or smoke can enter into the interior of the utility box where the monitoring system is located. In some examples, the cover may detach from the monitoring system. In the same or different examples, the cover may be paintable.

According to these examples, the monitoring system may blend better into the environment. For example, because it is recessed into a utility box, a smoke detector may no longer take up room in the room. For monitoring systems with a cover, the cover may be decorative and paintable so that it can blend into the décor of the room. In some examples, multiple cover styles (e.g., linear, clover, circle, among others) may be offered. This may create an incentive for replacing existing (unsightly) monitoring systems with the attractive monitoring systems of the present disclosure.

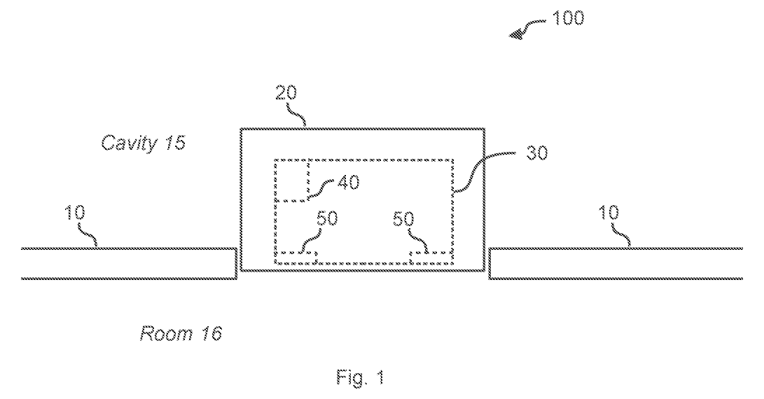

FIG. 1 illustrates an example monitoring system 100 according to the present disclosure. In the illustrated example, a detector fits within, and is installed inside of a utility box located in a ceiling cavity in a building. In some examples, the monitoring system (e.g., carbon monoxide detector / smoke detector) is secured within the utility box via attachment means provided by the utility box. For example, screws may attach the monitoring system to the utility box using pre-existing screw receiver holes in the utility box. In another example, the monitoring system may be equipped with clips that clip into pre-existing clip receiver locations in the utility box or clips that clip onto one or more the utility box housing members. In the same or different examples, the monitoring system may be connected to pre-existing wires (e.g., those used for the unsightly monitoring systems). In the same or different example, the pre-existing wires may be connected to monitoring system via a wiring assembly that is part of or connected to the monitoring system.

The example monitoring system 100 depicted in section view FIG. 1 may include utility box 20, detector 30, and ceiling 10. In some examples, detector 30 may include a smoke sensor which may be configured to detect the presence of smoke particles in the air. In some examples, the detector 30 may utilize photoelectric or ionization technology to detect smoke. In the same or different examples, detector 30 may include a carbon monoxide sensor which may be designed to detect the presence of carbon monoxide gas in the surrounding environment. In some examples, detector 30 may use electrochemical or semiconductor sensor technology to detect carbon monoxide levels.

In the same or different examples, detector 30 may include an alarm (not depicted) that may be connected to one or both of a smoke sensor and a carbon monoxide sensor. In some examples, the alarm may be configured to generate an audible or visual alert when the smoke sensor or the carbon monoxide sensor detects a potential hazard. The alarm may include a speaker, LED lights, or other signaling devices to alert occupants of the monitored space.

As depicted in FIG. 1, utility box 20 may be recessed within a cavity 15 behind ceiling 10 so that it does not protrude into room 16. In an example, detector 30 may include attachment assembly 50 and wiring assembly 40 (shown in dotted lines because they may fit substantially inside utility box 20). For example, detector 30 may be sized to occupy the interior space of utility box 20 without protruding beyond the surface of ceiling 10. This configuration may allow for a low-profile installation that integrates seamlessly with existing building infrastructure. In some cases, utility box 20 may be rectangular, octagonal, circular, or any other shape, providing flexibility for installation in various building structures (including building structures with pre-existing utility boxes).

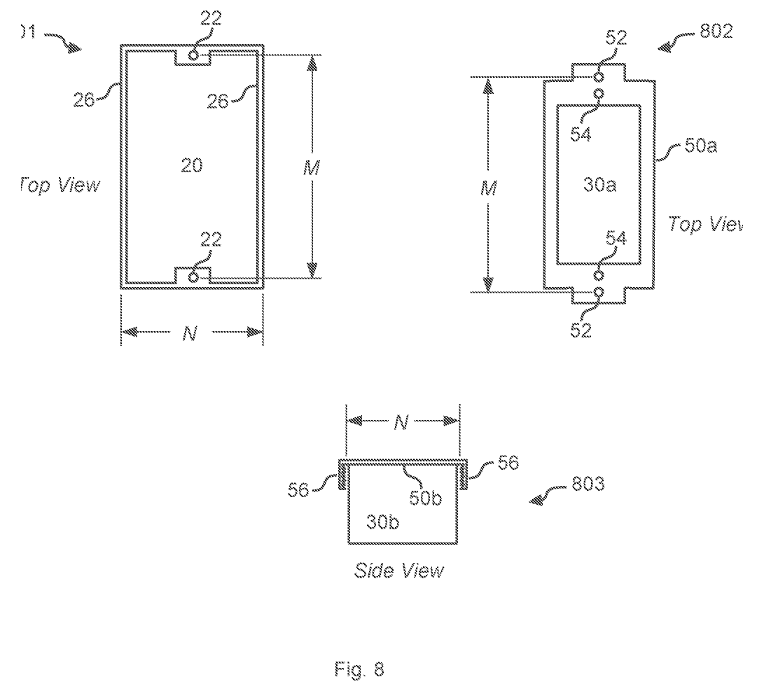

In an example, attachment assembly 50 may be used to secure detector 30 within utility box 20. Attachment assembly 50 may be any structure sufficient to facilitate the connection between detector 30 and utility box 20, ensuring a stable and reliable installation. Attachment assembly 50 may be integral with detector 30 or may be constructed as a separate structure that attaches (removably or permanently) to detector 30. In some cases, attachment assembly 50 may include screw holes configured to align with pre-existing screw receiver holes in utility box 20 (see FIG. 8). This alignment may allow for straightforward installation using standard fasteners, potentially simplifying the mounting process.

In an example, wiring assembly 40 may include any structure sufficient to connect power wires and any necessary communication wires to the appropriate terminals on detector 30. Wiring assembly 40 may be integral with detector 30 or may be constructed as a separate structure that attaches (removably or permanently) to detector 30.

The installation configuration depicted in FIG. 1 may demonstrate how monitoring system 100 utilizes the existing utility box 20 as an external housing. By leveraging this pre-existing infrastructure, monitoring system 100 may offer a streamlined installation process that minimizes modifications to the building structure. In some implementations, this approach may reduce installation time and complexity compared to traditional protruding detector installations.

The recessed nature of detector 30 within utility box 20, as shown in FIG. 1, may provide aesthetic benefits. In some cases, this configuration may result in a less obtrusive appearance compared to conventional detectors that extend from the ceiling surface. The low-profile design may blend more seamlessly with the surrounding ceiling, potentially enhancing the overall visual appeal of the space while maintaining the functional aspects of the monitoring system.

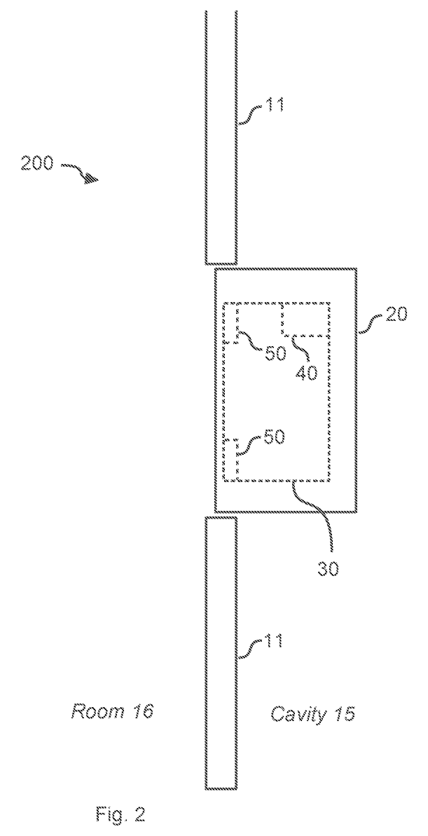

FIG. 2 illustrates an example monitoring system 200 according to the present disclosure. Monitoring system 200 is the same compared to example monitoring system 100 (FIG. 1) except that utility box 20 may be recessed within a cavity 15 behind wall 11 so that it does not protrude into room 16. In contrast, utility box 20 in FIG. 1 is recessed within a cavity 15 behind ceiling 10. Thus, some examples of the present disclosure provide systems and methods for monitoring systems installed in ceilings whereas other examples of the present disclosure provide systems and methods for monitoring systems installed in walls.

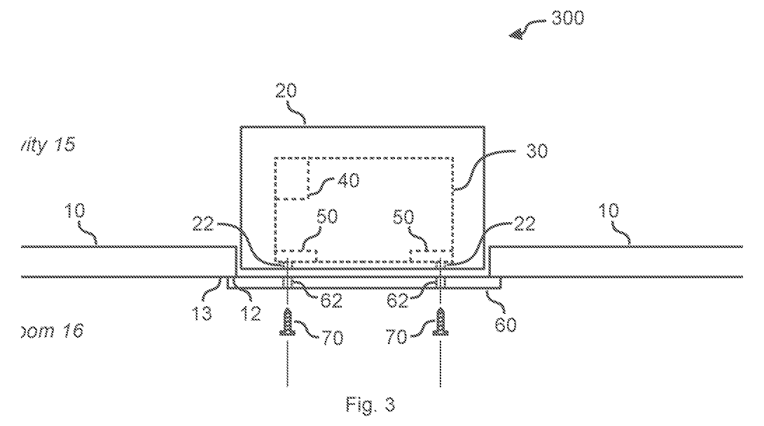

FIG. 3 illustrates an example monitoring system 300 according to the present disclosure. FIG. 3 is similar to FIG. 1, with the addition of monitoring system cover 60. In the depicted example, surface 12 of cover 60 may be flush with ceiling surface 13 of ceiling 10. For example, cover 60 may attach to detector 30 or utility box 20 with screws 70 or clips (not illustrated) that tighten cover 60 to ceiling surface 13. In this way, surface 12 of cover 60 may form a substantially continuous seal with ceiling surface 13. In the illustrated example, cover 60 may include holes 62 through which screws 70 may be inserted. In the same or different examples, utility box 20 may have holes 22 through which screws 70 may be inserted to fasten cover 60 to the assembly.

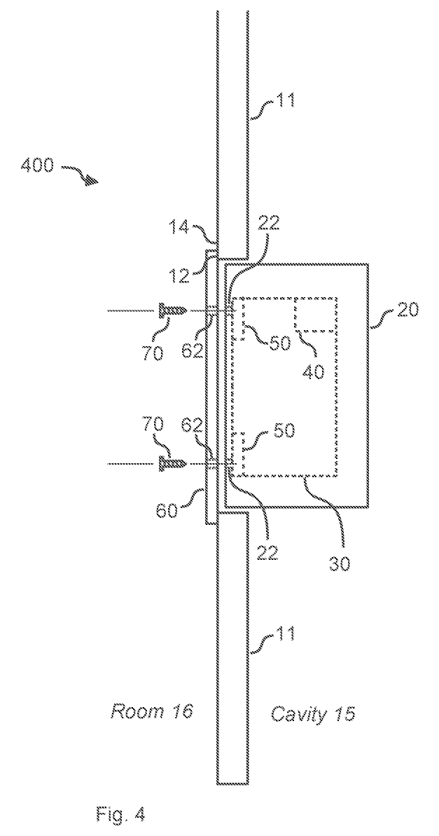

FIG. 4 illustrates an example monitoring system 400 according to the present disclosure. Monitoring system 400 is the same compared to example monitoring system 300 (FIG. 3) except that utility box 20 may be recessed behind wall 11 instead of behind ceiling 10 (FIG. 3). In the illustrated example, surface 12 of cover 60 may be flush with wall surface 14 of wall 11. In this way, surface 12 of cover 60 may form a substantially continuous seal with wall surface 14.

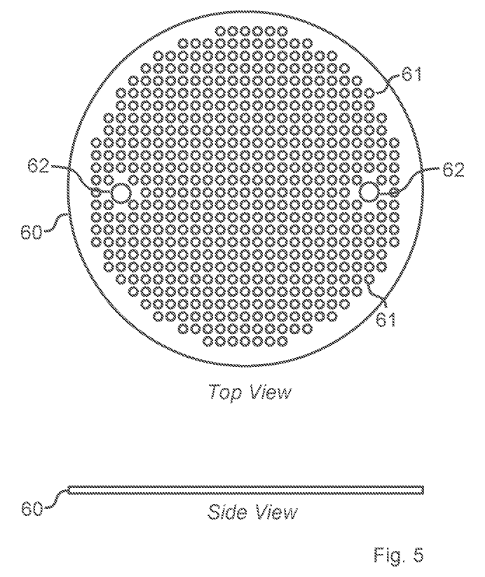

FIG. 5 illustrates an example cover 60 (top and side views) to be used with various examples of the present disclosure. Cover 60 may be positioned between the interior space in a room (e.g., room 16 in FIGS. 1-4) and the utility box 20. Cover 60 may serve both aesthetic and functional purposes. In some examples, cover 60 may include ventilation openings 61 that allow airflow between the interior space in room 16 and the inside of a utility box (e.g., utility box 20 in FIG. 1). This airflow may be important for the proper functioning of the monitoring system (e.g., smoke sensor, carbon monoxide sensor, among others). The ventilation openings 61 may be designed to allow sufficient air movement while maintaining the aesthetic appeal of cover 60. Cover 60 may also include holes 62 through which screws 70 (FIGS. 3-4) may be inserted. Cover 60 may be designed to attach to detector 30 or utility box 20, providing a finished appearance to the installed monitoring system.

In the example illustrated in FIG. 5, openings 61 may be circular holes. In some cases, the cover 60 may include other various design options including openings 61 that have different shapes (squares, diamonds, zigzag, among others). Thus, in some examples cover 60 may be customized with different patterns or styles to match the décor of the monitored space. The surface of cover 60 may be designed to be paintable, allowing for further customization of cover 60 to blend with the surrounding environment. In some examples, the cover 60 may be detachable from detector 30 or utility box 20. This detachable design may facilitate maintenance, inspection, painting, or replacement of detector 30 without requiring complete removal of the detector 30 from its installed location.

In some examples, cover 60 may also accommodate additional customization options. In some implementations, a decal may be applied to cover 60 to match specific colors, styles, or wood finishes. This decal option may provide a cost-effective method for customizing the appearance of monitoring system 300 without requiring multiple cover assembly designs.

Example cover 60 is illustrated having a circular shape. In some examples, cover 60 may be rectangular, octagonal, circular, or any other shape, providing flexibility for installation in various building structures (including building structures with pre-existing utility boxes). In some examples, the shape of cover 60 may correspond to the shape of utility box 20. In other examples, the shape of cover 60 may be different from the shape of utility box 20.

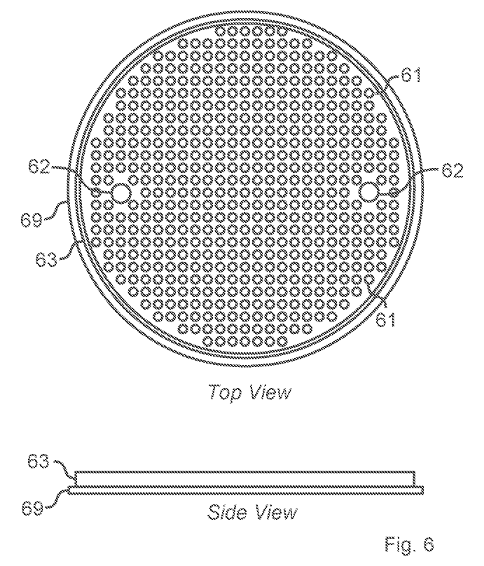

FIG. 6 illustrates an example cover 69 (top and side views) to be used with various examples of the present disclosure. Cover 69 is similar to cover 60 (FIG. 5) except that cover 69 may include gasket 63 which may be a raised portion protruding from one surface of cover 69. Gasket 63 may be any structure sufficient to form a substantially continuous seal between cover 69 and the utility box 20 to which cover 69 is installed. For example, gasket 63 may be made of the same or different material as cover 69. In some examples, gasket 63 may be made of a rigid material (e.g., plastic, metal, among others). In other examples, gasket 63 may be made of a non-rigid material (e.g., foam, rubber, among others). Gasket 63 may be integral with cover 60 or may be constructed as a separate structure that attaches (removably or permanently) to cover 60.

The seal formed between gasket 63 and utility box 20 need not be air tight, but may be sufficient to ensure that air flowing into utility box 20 through openings 61 does is directed to detector 30 instead of flowing into cavity 15 through openings that are commonly found between ceiling/wall panels and installed utility boxes. In the illustrated example, both cover 69 and gasket 63 are circular in shape, which may be sufficient for installing with a circular utility box. In other examples, cover 69 may be circular in shape and gasket 63 may be non-circular (e.g., square, rectangle, octagon, among others), which may be sufficient for installing with a non-circular utility box (e.g., square, rectangle, octagon, among others). In other examples, cover 69 may be non-circular and gasket 63 may be any other shape appropriate for forming a seal between cover 69 and utility box 20.

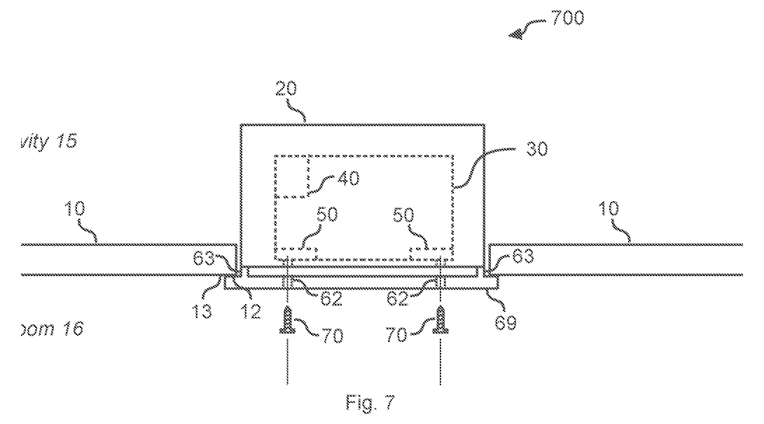

FIG. 7 illustrates an example monitoring system 700 according to the present disclosure. Monitoring system 700 is the same compared to example monitoring system 300 (FIG. 3) except that cover 69 with gasket 63 may be used in place of cover 60 without a gasket. In the illustrated example, gasket 63 of cover 60 may form a seal with utility box 20 when cover 69 is fastened to utility box 20 with screws 70. Although FIG. 7 depicts monitoring system 700 in ceiling cavity 15, cover 69 may also be used with a monitoring system installed in a wall cavity (e.g., as depicted in FIG. 4).

FIG. 8 illustrates various attachment assemblies for monitoring systems according to the present disclosure. At 801, utility box 20 may have pre-existing screw holes 22 and side walls 26. In the illustrated example, pre-existing screw holes 22 may be separated by distance M and utility box 20 may have width N. In the example illustrated at 802, detector 30a may have attachment assembly 50a. Attachment assembly 50a may include holes 52 configured to align with pre-existing screw holes 22 in utility box 20 (e.g., separated by distance M). In the same or different examples, attachment assembly 50a may include holes 54 configured to align with screw holes in cover 60 or 69 (e.g., holes 62 in FIGS. 4, 7). According to the example in 802, detector 30a may be installed into utility box 20 with screws through holes 52 and pre-existing screw holes 22. Cover 60 or 69 may then be installed with screws through holes 54 (in attachment assembly 50a) and holes 62 (in covers 60 and 69).

In the example illustrated at 803, detector 30b may have attachment assembly 50b. Attachment assembly 50b may include clips 56 configured to align with side walls 26 in utility box 20 (e.g., separated by distance N). In this example, detector 30 may be installed by pressing it into utility box 20 so that clips 56 grab onto side walls 26. In some examples, clips 56 may be configured to clip onto the outside of side walls 26 of utility box 20. In other examples, clips 56 may be configured to clip into the inside of side walls 26 (e.g., compression or spring-loaded). Although not depicted in 803, attachment assembly 50b may include holes (e.g., similar to holes 54) that may be configured to align with screw holes in cover 60 or 69 (i.e., for fastening cover 60 or 69 to attachment assembly 50b).

Although depicted with rectangular shapes, attachment assemblies 50a and 50b may be any shape sufficient to attach to various-shaped utility boxes (e.g., square, circle, rectangle, octagon, among others).

Attachment assemblies 50a and 50b may be integral with detectors 30a and 30b, respectively. Alternatively, attachment assemblies 50a and 50b may be constructed as a separate structures that attach (removably or permanently) to detectors 30a and 30b, respectively. In an example, providing attachment assemblies 50a and 50b that are separate structures configured to removably attach to detectors 30a and 30b allows for easier on-site installation where differently shaped utility boxes may be encountered. In this example, installers may easily and efficiently attach the correctly shaped attachment assembly to the detector based on the shape of any particular utility box.

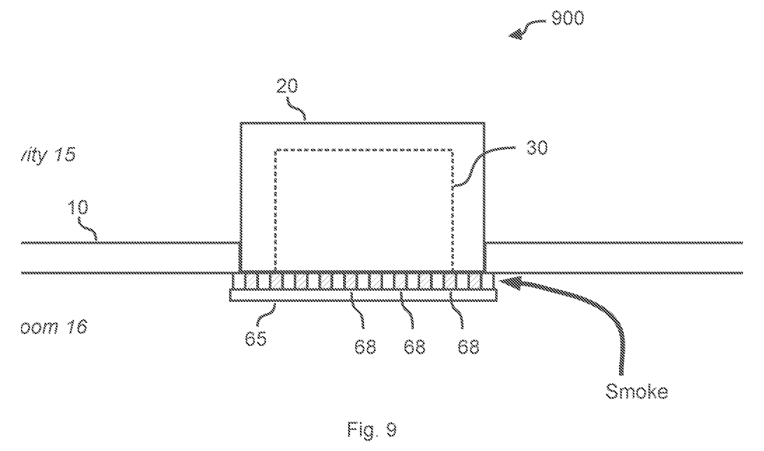

FIG. 9 illustrates an example monitoring system 900 according to the present disclosure. FIG. 9 is similar to FIG. 1, but includes cover 65 with air gaps 68 that allow for additional air (smoke) flow into the interior of utility box 20 where detector 30 is located.

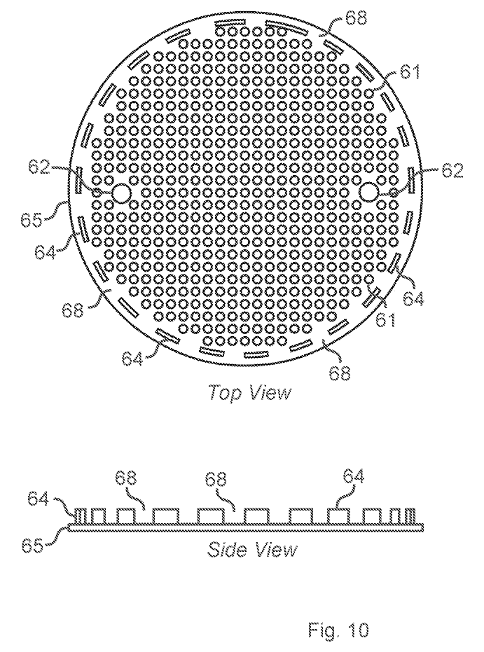

FIG. 10 illustrates top and side views of example cover 65 (FIG. 9) to be used with various examples of the present disclosure. Cover 65 is similar to cover 60 (FIG. 5) except that cover 65 may include horizontal air gap ring 64 which may be a raised portion protruding from one surface of cover 65. Horizontal air gap ring 64 may be configured as a broken ring so that it forms air gaps 68.

Example cover 65 is illustrated having a circular shape. In some examples, cover 65 may be rectangular, octagonal, circular, or any other shape, providing flexibility for installation in various building structures (including building structures with pre-existing utility boxes). In some examples, the shape of cover 65 may correspond to the shape of utility box 20. In other examples, the shape of cover 65 may be different from the shape of utility box 20. In some examples, the shape of horizontal air gap ring 64 may correspond to the shape of utility box 20. In other examples, the shape of horizontal air gap ring 64 may be different from the shape of utility box 20. Although cover 65 and horizontal air gap ring 64 are depicted having the same shape (circular), cover 65 may and horizontal air gap ring 64 may have shapes that differ from each other (e.g., square, circle, rectangle, octagon, among others).

In the example illustrated in FIG. 10, cover 65 does not include a gasket (e.g., gasket 63 in FIG. 6). In other examples (not illustrated), cover 65 may include both a horizontal air gap ring 64 and a gasket.

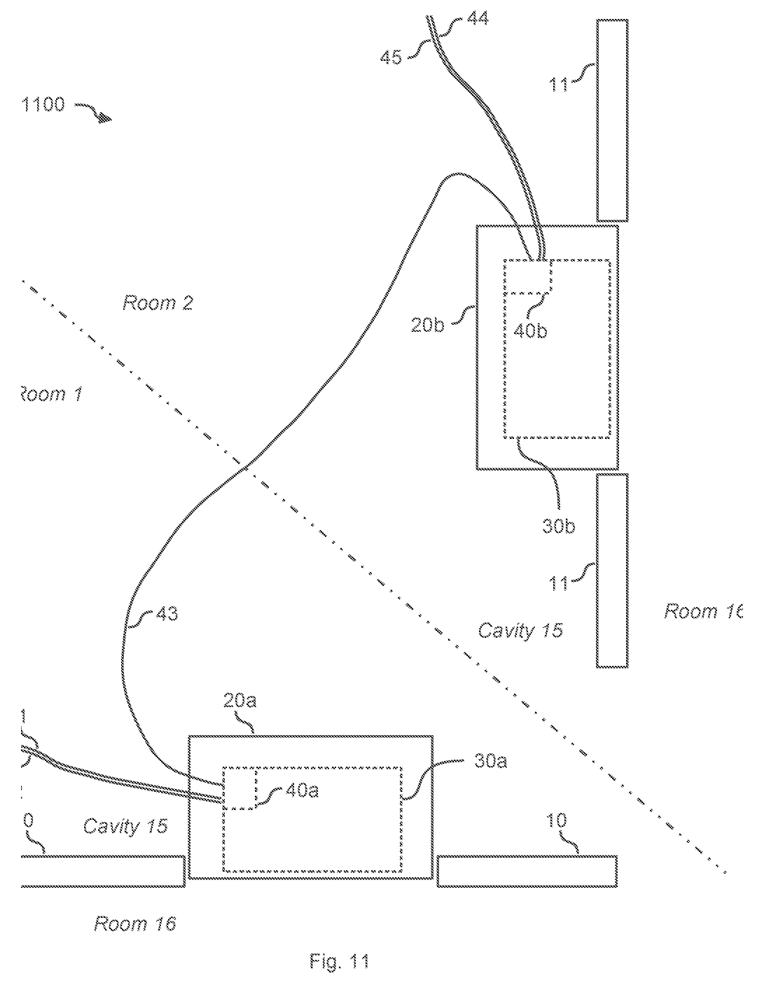

FIG. 11 illustrates an example monitoring system 1100 according to the present disclosure. In the depicted example, one monitoring system is installed in a wall cavity of Room 2 and another monitoring system is installed in a ceiling cavity of Room 1. According to the example, the Room 1 monitoring system may include utility box 20a and monitoring system 30a having wiring assembly 40a. In the same example, the Room 2 monitoring system may include utility box 20b and monitoring system 30b having wiring assembly 40b. Monitoring system 30a in Room 1 may be connected to a power source (not depicted) by wires 41 and 42 that may be coupled to wiring assembly 40a. Similarly, monitoring system 30b in Room 2 may be connected to a power source (not depicted) by wires 44 and 45 that may be coupled to wiring assembly 40b. In an example, wires 41/42 and 44/45 may be ROMEX or other wire sufficient to connect to the building’s power source (directly or indirectly). In the same or different examples, monitoring system 30b in Room 2 may connect to monitoring system 30a in Room 1 via wire 43. Wire 43 may connect to wiring assembly 40b in Room 2 and to wiring assembly 40a in Room 1. Wire 43 may be ROMEX or other wire sufficient to electrically connect monitoring systems 30a and 30b.

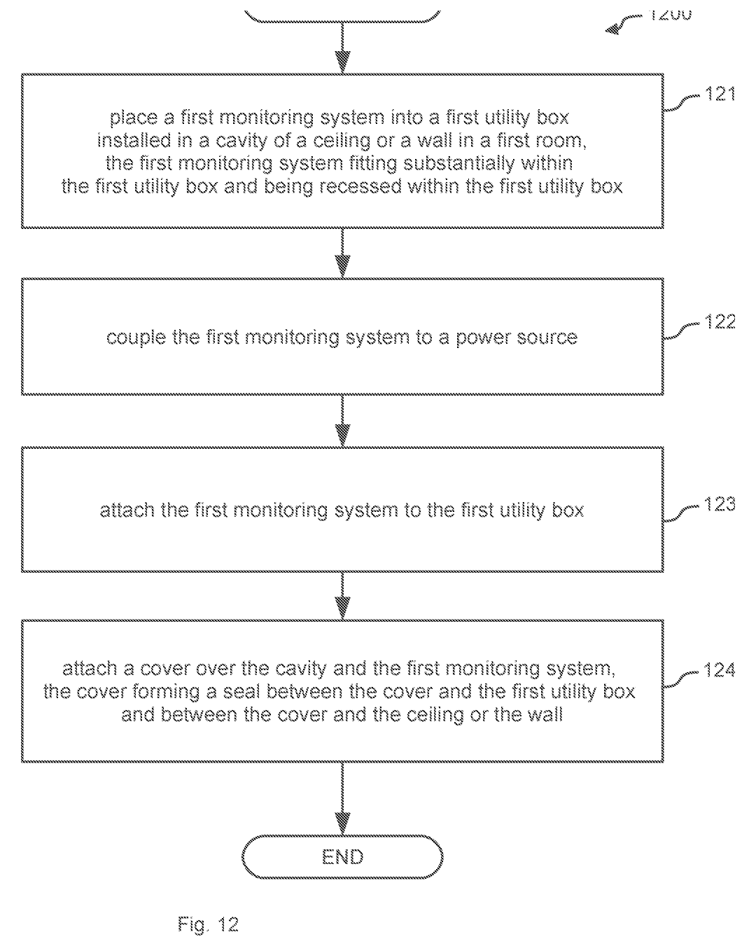

FIG. 12 illustrates a flow chart of an example method 1200 for recessing monitoring systems. According to one example, method 1200 may begin at block 1210. Teachings of the present disclosure may be implemented in a variety of configurations of monitoring system 100. As such, the initialization point for method 1200 and the order of 1210-1240 comprising method 1200 may depend on the implementation chosen.

At block 1210, a first monitoring system may be placed into a first utility box installed in a cavity of a ceiling or a wall in a first room so that the first monitoring system may fit substantially within the first utility box and be recessed within the first utility box. At block 1220, the first monitoring system may be coupled to a power source. At block 1230, the first monitoring system may be attached to the first utility box. In an example, the attaching the first monitoring system to the first utility box may include using pre-existing screw holes in the first utility box to secure the first monitoring system to the first utility box. At block 1240, a cover may be attached over the cavity and the first monitoring system so that the cover may form a seal between the cover and the first utility box and between the cover and the ceiling or the wall. In some examples, the attaching the cover over the cavity and the first monitoring system may include snapping the cover onto one of the first utility box and the first monitoring system. For example, the cover may include clips, snaps, or other attachment assemblies that snap onto the first utility box or the first monitoring system when pressed against either structure.

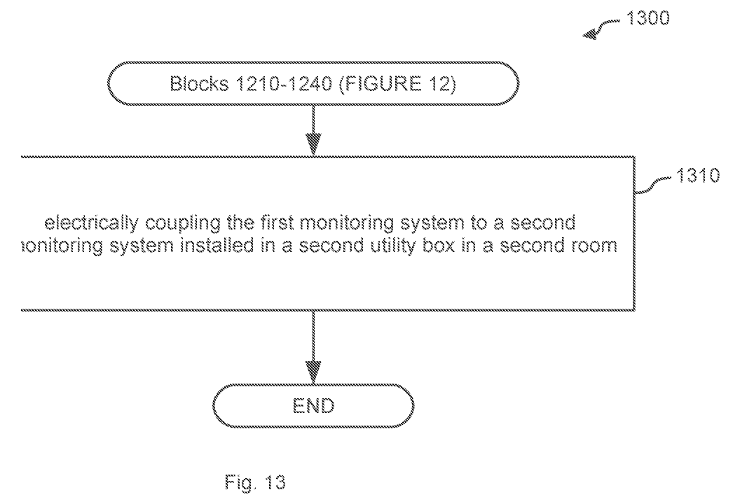

Although FIG. 12 discloses a particular number of operations related to method 1200, method 1200 may be executed with greater or fewer operations than those depicted in FIG. 12. For example, after block 1240, method 1200 may continue with additional operations illustrated in FIG. 13. In addition, although FIG. 12 discloses a certain order of operations to be taken with respect to method 1200, the operations comprising method 1200 may be completed in any suitable order.

FIG. 13 illustrates a flow chart of an example method 1300 for recessing monitoring systems. According to one example, method 1300 may begin at block 1310. Teachings of the present disclosure may be implemented in a variety of configurations of monitoring system 100. As such, the initialization point for method 1300 and the order of 1310 comprising method 1300 may depend on the implementation chosen.

Method 1300 may begin with blocks 1210-1240 (FIG. 12) and then proceed to block 1310. At block 1310, the first monitoring system may be electrically coupled to a second monitoring system installed in a second utility box in a second room.

Although FIG. 13 discloses a particular number of operations related to method 1300, method 1300 may be executed with greater or fewer operations than those depicted in FIG. 13. In addition, although FIG. 13 discloses a certain order of operations to be taken with respect to method 1300, the operations comprising method 1300 may be completed in any suitable order. For example, block 1310 may occur at any time after block 1210.

Methods 1200-1300 may be implemented using monitoring system 100 or any other system operable to implement methods 1200-1300. Although examples have been described above, other variations and examples may be made from this disclosure without departing from the spirit and scope of these disclosed examples

Thus, a utility box form fitted detector with a stylish, paintable, and exchangeable cover may be provided. Changes may be made to the present disclosure without departing from the spirit and scope of the disclosure.

1. An apparatus, comprising:
a detector configured to fit substantially within a utility box installed in a cavity of a ceiling or a wall in a room;
a wiring assembly configured to couple the detector to a power source; and
an attachment assembly configured to attach the detector to the utility box,
wherein the detector is recessed within the utility box when attached to the utility box.

2. The apparatus of claim 1, comprising:
a cover configured to attach to the utility box,
wherein a surface of the cover is configured to be substantially flush with a ceiling surface of the ceiling or a wall surface of the wall when the cover is attached to the utility box.

3. The apparatus of claim 2, wherein the cover comprises one or more openings to allow airflow from the room into the utility box.

4. The apparatus of claim 3, wherein the cover comprises a gasket configured to form a seal between the cover and the utility box.

5. The apparatus of claim 4, wherein the cover comprises a plurality of holes configured to align with a plurality of pre-existing screw holes in the utility box.

6. The apparatus of claim 2, wherein the cover is detachable from the utility box.

7. The apparatus of claim 1, wherein the attachment assembly comprises a plurality of holes configured to align with a plurality of pre-existing screw holes in the utility box.

8. The apparatus of claim 1, wherein the attachment assembly comprises a clip configured to clip onto the utility box.

9. The apparatus of claim 1, wherein the detector comprises at least one of a smoke detector and a carbon monoxide detector.

10. A system, comprising:
a utility box installed in a cavity of a ceiling or a wall in a room;
a first monitoring system configured to fit substantially within the utility box, the first monitoring system comprising:
a wiring assembly configured to couple the first monitoring system to a power source;
an attachment assembly configured to attach the first monitoring system to the utility box, wherein the first monitoring system is recessed within the utility box when attached to the utility box; and
a cover comprising one or more openings to allow airflow from the room into the utility box, wherein the cover is configured to:
detachably attach to one of the first monitoring system, the ceiling, the wall, and the utility box;
form a seal between the cover and the utility box; and
overlap and be substantially flush with a ceiling surface of the ceiling or a wall surface of the wall when attached to the one of the first monitoring system, the ceiling, the wall, and the utility box.

11. The system of claim 10, wherein a first surface of the cover is in contact with the ceiling surface or the wall surface.

12. The system of claim 11, wherein the first surface of the cover forms a substantially continuous seal with the ceiling surface or the wall surface.

13. The system of claim 10, wherein the cover comprises a plurality of holes configured to align with a plurality of pre-existing screw holes in the utility box.

14. The system of claim 10, wherein the attachment assembly comprises a plurality of holes configured to align with a plurality of pre-existing screw holes in the utility box.

15. The system of claim 10, wherein the attachment assembly comprises a clip configured to clip onto the utility box.

16. The system of claim 10, wherein the first monitoring system comprises at least one of a smoke detector and a carbon monoxide detector.

17. The system of claim 10, wherein the wiring assembly is configured to couple the first monitoring system to a second monitoring system.

18. A method, comprising:
placing a first monitoring system into a first utility box installed in a cavity of a ceiling or a wall in a first room, the first monitoring system fitting substantially within the first utility box and being recessed within the first utility box;
coupling the first monitoring system to a power source;
attaching the first monitoring system to the first utility box; and
attaching a cover over the cavity and the first monitoring system, the cover forming a seal between the cover and the first utility box and between the cover and the ceiling or the wall.

19. The method of claim 18, wherein the attaching the first monitoring system to the first utility box comprises using pre-existing screw holes in the first utility box to secure the first monitoring system to the first utility box.

20. The method of claim 19, wherein the attaching the cover over the cavity and the first monitoring system comprises snapping the cover onto one of the first utility box and the first monitoring system.

21. The method of claim 18, comprising: electrically coupling the first monitoring system to a second monitoring system installed in a second utility box in a second room.
